(19) United States
(12) Plant Patent Application Publication
(10) Pub. No.: US 2003/0056256 P1
(43) Pub. Date: Mar. 20, 2003

(54) Grapevine '9-9-12'

(76) Inventor: Harold P. Olmo, Davis, CA (US)

Correspondence Address: Harold P. Olmo, 6964 Vineyard Lane, Davis, CA, 95616, US

(21) Appl. No.: 09/901,870

(22) Filed: Jun. 26, 2001

(51) Int. Cl. A01H 5/00

(52) U.S. Cl. PLT/207

(57) ABSTRACT

A new and distinct variety 9-9-12 of the vinifera grape, comparable to Thompson Seedless as a dessert fruit and of similar quality for fresh consumption. The improvements in this invention appear to be very large berry size and more attractive berries after gibberellin treatment, and as well as extension of the harvesting season of fresh seedless grapes.

### PLANT CHARACTERISTICS

The important characteristics of the new Variety 9-9-12 have been observed since the first year of fruiting in 1994 and later when established vines in a trial plot at McFarland, Calif. The location is in a commercial table grape production zone. Vines were established by grafting to several rootstocks and notes taken for three consecutive years, 1998-2000. Several vines were also propagated on their own roots from cuttings. The characters of these vines remained uniform and comparable with those present in the original selection.

This invention describes a new and distinct variety of grapevine of the vinifera species that produces very large seedless berries and ripens later than the standard Thompson Seedless, thus extending the harvest season of fresh table grapes of high dessert quality.

### IN THE DRAWINGS

FIG. 1 Fruit clusters at harvest time, natural and gibberellin treatment.

FIG. 2 Leaf, upper surface.

FIG. 3 Leaf, lower surface.

Vine: Large, vigorous and long canes, lateral branching, limited or only weak, fruit clusters pendant from canes, tendrils weak, canopy open. Budding out 5 to 6 days after Thompson Seedless with similar response to current pests and diseases.

Shoot Tip (10″-12″): Glabrous, first unfolding leaf (flattened), three lobed, shiny surface, coppery colored, tendrils often trifid but thin and long.

Shoot: Inflorescences borne on nodes 4 or 5, but regularly only one per shoot. Cane pruning necessary for good yield on expanded trellising. Bud break from canes usually very high and uniform permitting yield control by deshooting.

Leaf: Large, cuneate in outline, 3-lobed, but upper sinuses very narrow and deep, obscured by overlapping lobes, striations on central lobe obtuse or absent, in very irregular sequence, petiolar sinus wide U-shaped; petiole slender, flushed pink.

Cane: Straight, with long internodes, dark reddish-brown striations of bark, contrasted with small and colorless leaf scar, lateral branching absent or weak, providing open canopy.

Cluster: Mature fruit cluster (natural), about 480 gm. short conical, 17 cm. in length, peduncle straight, often pendant, tendril with few berries 5 cm. from attachment plus 7-8 cm. to first fruiting lateral, sqme color as berries, average 115-120 berries per cluster.

Berry: Ellipsoidal, light green with white waxy bloom, on surface, gibberellin treated clusters retain features of untreated (natural) fruit, but almost double the berry size. Mean for 10 largest berries per cluster, natural 57.gm., treated 106.gm. Most berries have one or two rudimentary seeds that remain soft and reduced by treatment.

We claim:

1. The new and distinct variety of TABLE GRAPE herein described and illustrated and identified by the characteristics enumerated above.
